(19) United States
(12) Plant Patent Application Publication
(10) Pub. No.: US 2011/0277197 P1
(43) Pub. Date: Nov. 10, 2011

(54) Grapevine named 'Green Onyx'

(50) ;

(75) Inventors: Avichai Perl, Rishon Lezion (IL); Nachman Sahar, Rehovot (IL); Oded Degani, Moshav Sde Moshe (IL); Tatiana Okun, Rehovot (IL); Arie Sarfian, Nes-Ziona (IL); Hanan Bazak, Carmei Yossef (IL); Refael Eliassi, Neve Yamin (IL); Ahuva Daos, Ganey Hmoshava Mazkeret Batia (IL)

(73) Assignee: State of Israel, Ministry of Agriculture & Rural Development, Agricultural Research Organization

(21) Appl. No.: 12/929,129

(22) Filed: Jan. 3, 2011

(30) Foreign Application Priority Data
Jan. 4, 2010 (IL) 4236

(51) Int. Cl. A01H 5/00

(52) U.S. Cl. PLT/207

(57) ABSTRACT

This invention relates to a new and distinct variety of grapevine named ‘GREEN ONYX’. The new early ripening, very fertile grapevine variety is particularly characterized by an attractive berry color, a very uniform bunch and a firm berry with tender crunchy skin.

### PRIORITY CLAIM

This application claims priority under 35 U.S.C. §119(f) of the Israeli Plant Breeders' Rights Application No. 4236 filed Jan. 4, 2010.

### BOTANICAL NAME OF THE GENUS AND SPECIES OF THE PLANT CLAIMED

Vitis vinifera L.

### VARIETY DENOMINATION

‘GREEN ONYX’

### BACKGROUND OF THE INVENTION

The present invention relates to a new and distinct grapevine variety, botanically known as Vitis vinifera L., and hereinafter referred to by its variety denomination ‘GREEN ONYX’.

The new grapevine ‘GREEN ONYX’ is a product of a controlled breeding program conducted by the inventors, Avichai Perl, Nachman Sahar, Oded Degani, Tatiana Okun, Arie Sarfian, Hanan Bazak, Refael Eliassi and Ahuva Daos at the Agricultural Research Organization, The Volcani Center, located in Bet Dagan, Israel. The objective of the breeding program was to develop a new early ripening, very fertile grapevine variety particularly characterized by an attractive berry color, a very uniform bunch and a firm berry with tender crunchy skin.

The new grapevine ‘GREEN ONYX’ originated from a cross made by the inventors in 2004 at the Agricultural Research Organization, The Volcani Center. The female or seed parent is the grapevine variety, Vitis vinifera L indicated as ‘ARO 2117’ (unpatented), in the inventor's collection located in Bet Dagan, Israel, and the male or pollen parent is the grapevine variety, Vitis vinifera L. indicated as ‘ARO 1717’ (unpatented), in the inventor's collection located in Bet Dagan, Israel.

The new grapevine ‘GREEN ONYX’ was observed and selected by the inventors within the progeny of the stated cross in a controlled environment in 2007 at the Agricultural Research Organization, The Volcani Center.

Asexual propagation of the new grapevine variety by bud grafting was first performed in January 2008 at the Agricultural Research Organization, The Volcani Center, and has demonstrated that the combination of characteristics as herein disclosed for the new cultivar are firmly fixed and retained through successive generations of asexual propagation. The new cultivar propagates true to type.

### SUMMARY OF THE INVENTION

‘GREEN ONYX’ can be grown in various climates and growing conditions. The variety produces and maintains a strong vigorous plant and consistent fruit production from End June through End July in Bet Dagan, Israel.

The following traits have been repeatedly observed and are determined to be characteristics of ‘GREEN ONYX’, which in combination distinguish this grapevine as a new, unique and distinct variety.

1. Very fertile;

2. Early ripening;

3. Firm berry with tender crunchy skin;

4. Attractive berry color; and

5. A very uniform bunch.

Plants of the new grapevine ‘GREEN ONYX’ differ from plants of the parents, Vitis vinifera L. ‘ARO 2117’ (unpatented) and Vitis vinifera L. ‘ARO 1717’ (unpatented), in the following characteristics described in Table 1.

TABLE 1
Comparison with parent varieties.

| | | | |
|---|---|---|---|
| | | Female Parent | Male Parent |
| | New Variety | ‘ARO 2117’ | ‘ARO 1717’ |
| Characteristic | ‘GREEN ONYX’ | (unpatented) | (unpatented) |

Of the many commercial varieties known to the present inventors, the most similar to the new grapevine ‘GREEN ONYX’ are Vitis vinifera L. ‘SUGRAONE’ (patented) and Vitis vinifera L. ‘PRIME’ (patented) which differ from the new grapevine ‘GREEN ONYX’ in the characteristics described in Table 2:

TABLE 2
Comparison with similar varieties.

| | | | |
|---|---|---|---|
| | | Comparison | Comparison |
| | New Variety | Variety | Variety |
| | ‘VARIETY | ‘SUGRAONE’ | ‘PRIME’ |
| Characteristic | DENOMINATION’ | (patented) | (patented) |

### BRIEF DESCRIPTION OF THE PHOTOGRAPHS

The accompanying photographs illustrate the overall appearance of the new grapevine ‘GREEN ONYX’ showing the colors as true as is reasonably possible with colored reproductions of this type. Colors in the photographs may differ slightly from the color values cited in the detailed morphological description, which accurately describe the color of ‘GREEN ONYX’.

FIG. 1 shows a typical bunch of ‘GREEN ONYX’ on the plant.

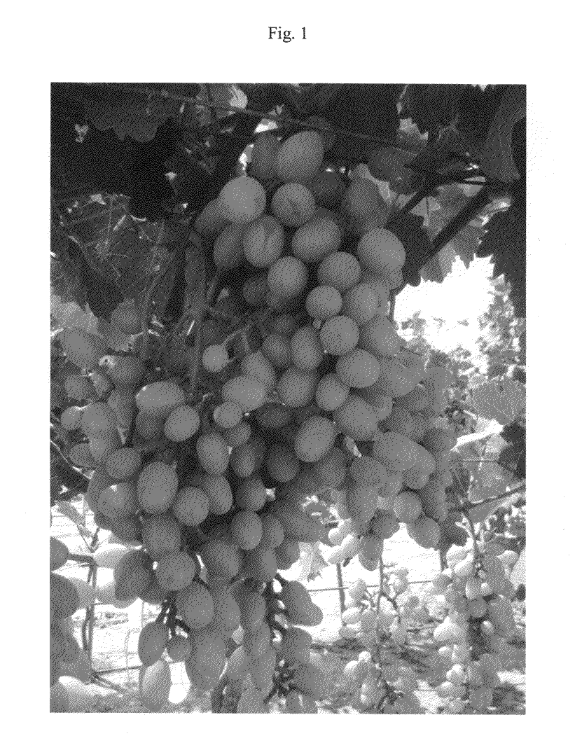

FIG. 2 shows a typical mature leaf of ‘GREEN ONYX’.

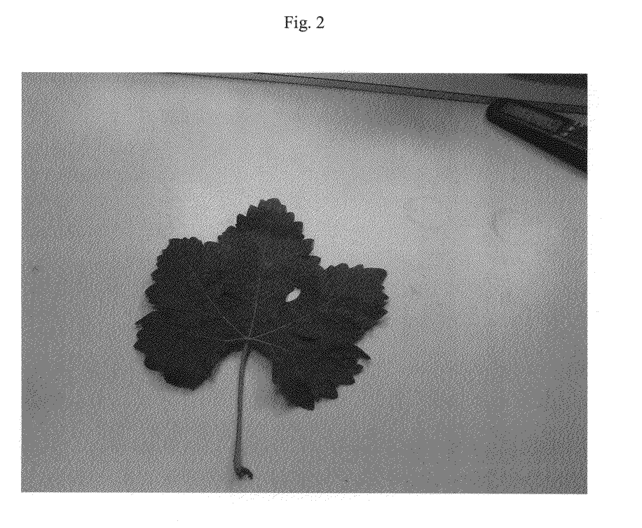

FIG. 3 shows a typical inflorescence of ‘GREEN ONYX’.

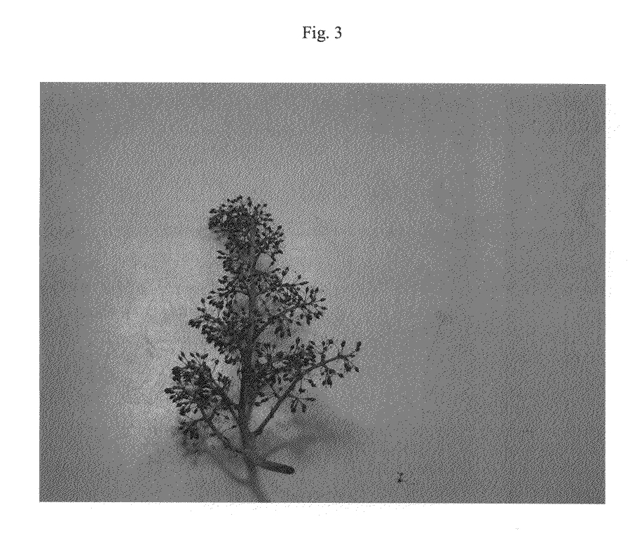

FIG. 4 shows a typical growth tip of ‘GREEN ONYX’.

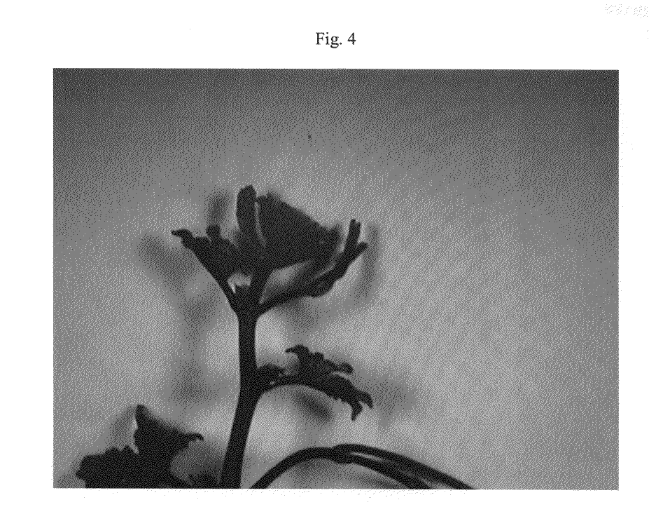

### DETAILED MORPHOLOGICAL DESCRIPTION

The new Vitis vinifera L. ‘GREEN ONYX’ has not been observed under all possible environmental conditions. The phenotypical descriptions and color designations stated for the new variety may vary, depending on variations in environmental factors, including weather (temperature, humidity and light intensity), day length, soil type, location and cultural conditions, without any change in the genotype of the grapevine.

The following description of ‘GREEN ONYX’ unless otherwise noted, is based on observations and measurements taken during 2010 and 2011 at the Agricultural Research Organization, The Volcani Center, on plants grown in the vineyard located in Bet Dagan, Israel under conditions which closely approximate those generally used in commercial practice. The described plants were grafted on ‘Richter110’ (unpatented) and planted at a distance of 1.5 m in sandy red loam soil at an elevation of about 30 meters above sea level, with drip-irrigation up to 40 m³ per hectare in the peak season in the summer and Shefer 737+microelements 1.5 ltr. fertilizers in each 5 m³ of water. Average annual rainfall is about 550 mm, with an average of 350 mm rainfall in winter (December through February). Mean diurnal minimum temperature in January is 7.2° C., and mean diurnal maximum temperature in July is 30.8° C.

Unless otherwise stated, the detailed morphological description includes observations, measurements and values based on three-year-old ‘GREEN ONYX’ plants grown in the vineyard at the Agricultural Research Organization, The Volcani Center, located in Bet Dagan, Israel from 2010 to 2011. Quantified measurements are expressed as an average of measurements taken from a number of plants of ‘GREEN ONYX’. The measurements of any individual plant, or any group of plants, of the new variety may vary from the stated average.

Color references are made to The Royal Horticultural Society Colour Chart (R.H.S.), 1986 edition, except where general colors of ordinary significance are used. Color values were taken under daylight conditions in full sunlight in Bet Dagan, Israel.

All of the plants of ‘GREEN ONYX’, insofar as they have been observed, have been consistent in all the characteristics described below.
Classification:
   Botanical.—Vitis vinifera L.
Parentage:
   Female or seed parent.—Vitis vinifera L. ‘ARO 2117’ (unpatented).
   Male or pollen parent.—Vitis vinifera L. ‘ARO 1717’ (unpatented).
Propagation: Grafting onto ‘Richter110’ (unpatented)
Growing conditions:
   Light intensities.—Full sunlight.
   Temperature.—Bet Dagan — 30 meters above sea level.

| Elements | January | February | March | April |
|---|---|---|---|---|
| Mean maximum air | 17.8 | 18.1 | 20.1 | 24.5 |
| temperature (deg C.) | | | | |
| Mean minimum air | 7.2 | 7.1 | 8.8 | 11.5 |
| temperature (deg C.) | | | | |
| Mean rainfall (mm) | 140.5 | 96.9 | 66.1 | 17.5 |
| Elements | May | June | July | August |
| Mean maximum air | 27 | 29.2 | 30.8 | 31.2 |
| temperature (deg C.) | | | | |
| Mean minimum air | 14.6 | 17.9 | 20.6 | 21.2 |
| temperature (deg C.) | | | | |
| Mean rainfall (mm) | 2.2 | — | — | — |
| Elements | September | October | November | December |
| Mean maximum air | 30.4 | 28.3 | 24.1 | 19.7 |
| temperature (deg C.) | | | | |
| Mean minimum air | 19.4 | 16 | 11.8 | 8.6 |
| temperature (deg C.) | | | | |
| Mean rainfall (mm) | 0.4 | 20.4 | 76.2 | 130.3 |

Fertilization.—A balanced fertilizer with level of Shefer 737 +microelements 1.5 ltr. in each 5 m³ of water.
Growth regulators.—None applied.
Pruning.—as in commercial practice.
Training.—Y trailing cordon.
Plant.—Time of vegetative bud burst: N/R.
Young shoot.—Openness of tip: Closed. Density of prostrate hairs on tip: Dense. Anthocyanin coloration of prostrate hairs on tip: Very weak. Green color: Green RHS 144 A. Green color of upper side of blade: Medium green RHS 144 A with over color medium red brown RHS 166 CD. Green color of lower side of blade: Medium green RHS 144 A with over color medium red brown RHS 166 CD. Anthocyanin coloration: Medium.
Young leaf.—Density of hairs between main veins on lower side of blade: Medium. Glossiness of upper side: Strong. Glossiness of lower side: Weak.
Shoot.—Color of dorsal side of internode: N/R. Color of ventral side of internode: N/R. Color of dorsal side of node: N/R. Color of ventral side of node: N/R. Density of erect hairs on internodes: N/R. Number of consecutive tendrils: N/R. Length of tendril: N/R.
Infloresence.—Length: 16-25 cm. Maximum diameter: 8-10 cm. No. of side branches: 13-15. Attitude of side branches: Semi upright. Length of longest side branch: 5-6 cm. Thickness of main axis: About 4 mm. Green color of main axis: Light green RHS 144 B. Anthocyanin coloration of main axis: Weak. Pubesence of main axis: Absent.
Flower.—Sexual organs: Fully developed stamens and fully developed gynoecium.
Pedicel.—Length: 4-6 mm. Thickness: 1-2 mm. Green color: Light green RHS 144 B. Pubesence: Absent.
Stamen.—Number: Five. Length of filament: 3-4 mm. Thickness of filament: Minuscule. Pubesence of filament: Absent. Surface: Smooth. Color: light green RHS 145 C.
Anther.—Shape: Transverse elliptic. Length: Minuscule. Color: Light green RHS 145 C.
Pollen.—Color: Yellow RHS 150 B.
Ovary.—Shape: Elliptic. Color: Green RHS 144 A. Length: 2-3 mm. Diameter: 1-2 mm. Surface: Smooth.
Style.—Length: Minuscule.
Stigma.—Size: Minuscule. Color: Yellow RHS 150 B.
Mature leaf.—Size of blade: N/R. Blade length: 16-21 cm. Blade width: 17-20 cm. Shape of blade: Pentagonal. Green color of upper side: Dark green RHS 139 A. Green color of lower side: Dark green RHS 137 B. Glossiness of upper side: Medium. Glossiness of lower side: Medium. Profile in cross section: Undulated. Blistering of upper side: Medium. Number of lobes: Five. Central lobe length: About 11 cm. Central lobe width: About 9.5 cm. Upper, side lobe length: 7-9 cm. Upper, side lobe width: About 7 cm. Pubescence of veins on upper side: Sparse. Pubescence of veins on lower side: Medium. Green color of veins on upper side: Green RHS 137 C. Green color of veins on lower side: Yellow green RHS 145 A. Anthocyanin coloration of veins on upper side: Absent. Anthocyanin coloration of veins on lower side: Absent. Depth of upper lateral sinuses: Medium. Arrangement of lobes of upper leaf sinuses: Slightly overlapping. Arrangement of lobes of petiole sinus: Open. Petiole sinus limited by veins: Absent. Length of distal tooth: 10-16 mm. Width of distal tooth: 13-20 mm. Ratio length/width of distal tooth: Small. Shape of teeth: Straight on both sides. Anthocyanin coloration of main veins on upper side of blade: Absent. Density of hairs between veins on upper side of blade: Medium. Density of hairs between veins on lower side of blade: Medium to strong. Length of petiole compared to middle vein: Similar. Length of petiole: 11-14 cm. Petiole thickness: 4-6 mm.
Petiole.—Green color of upper side: Green yellow RHS 145 A. Anthocyanin coloration of upper side: Absent. Green color of lower side: Green yellow RHS 145 A. Anthocyanin coloration of lower side: Weak. pubescence: weak.
Fruit.—Time of beginning of berry ripening: N/R.
Bunch.—Size (peduncle excluded): N/R. Density: N/R. Length of peduncle: N/R.
Berry.—Size: N/R. Shape in profile: Broad elliptic. Color of skin (without bloom): Yellowish green. Ease of pedicel detachment: N/R. Thickness of skin: N/R. Flesh anthocyanin coloration: weak. Firmness of flesh: N/R. Juiciness of flesh: N/R. Particular flavour: Aromatic. Formation of seeds: Absent.
Woody shoot.—Main color (without bloom): N/R. Relief of surface: N/R.
Usage: Table grape.
Disease\pest resistance: No atypical resistance has been noted.

What is claimed is:

1. A new and distinct grapevine variety, referred to as ‘GREEN ONYX’, as herein described and illustrated by the characteristics set forth above.
